(19) United States
(12) Patent Application Publication
(10) Pub. No.: US 2004/0185444 A1
(43) Pub. Date: Sep. 23, 2004

(54) System for predicting susceptibility of human oropharyngeal squamous cell carcinoma to radiotherapy

(76) Inventors: Nicole Boehringer-Wyss, Geneva (CH); Stuart G. Clarkson, Geneva (CH); Abdelkarim S. Allal, Carouge (CH)

Correspondence Address: John Moetteli, Esq., Moetteli & Associe, SaRL, Case Postale 486, Geneva 12, CH-1211, CH

(21) Appl. No.: 10/391,438

(22) Filed: Mar. 18, 2003

(51) Int. Cl. C12Q 1/68; G01N 33/574

(52) U.S. Cl. 435/6; 435/7.23

(57) ABSTRACT

The present invention is a gene expression system for prognosticating susceptibility of a human oropharyngeal squamous cell carcinoma (SCC) to radiotherapy. A selection of human gene fragments is disclosed which are useful, when utilized in a gene expression procedure, for detecting nucleotide sequences in a human SCC sample the over/under expression of which is predictive of the SCC's susceptibility to radiotherapy. Further, a sub-selection of five genes (FLJ11342, H08808, TOP1, DLD and EIF4A2) is identified, which showed a particularly distinct pattern wherein all susceptible (controlled) tumor biopsies were under-expressed and all unsusceptible (not-controlled) tumor samples were over-expressed.

### FIELD OF THE INVENTION

The present invention is in the field of organic compounds useful for testing susceptibility of cancer cells to radiotherapy. More specifically, the present invention relates to an array of human gene fragments useful in detecting nucleotide sequences in a human tumor biopsy sample, which nucleotide sequences are predictive of the tumor's susceptibility or response to radiotherapy.

Abbreviations

RT: radiotherapy

SCC: squamous cell carcinoma

hnSCC: head and neck squamous cell carcinoma;

C: controlled, susceptible/responsive to RT

NC: not-controlled, not susceptible/responsive to RT

T: test

EST: expressed sequence tag

TOP1: topoisomerase 1

DLD: dihydrolipoamide dehydrogenase

EIF4A2: eukaryotic translation initiation factor 4A, isoform 2

MMP: matrix metalloproteinase;

UPA: urokinase plasminogen activator

uPAR: UPA receptor

PAI-1: plasminogen activator inhibitor-1

TGF-β: transforming growth factor β.

### BACKGROUND OF THE INVENTION

In patients with head and neck carcinomas, as in the majority of cancer patients, indicators of oncological outcome are based traditionally on clinical and pathological features such as tumor size/stage, extent of lymph node involvement, and anatomical subsite. However, even after careful evaluation of these factors, predicting the clinical outcome remains hazardous, particularly for tumors of the same stage and sublocation. Moreover, patients with advanced disease are often treated with a combination of radiotherapy (RT) and chemotherapy and many of them suffer from the acute toxicity without commensurate benefit from such combination regimens. Thus, there is a clear need to identify additional prognostic factors, which can differentiate between those patients that potentially can benefit from combining RT with chemotherapy, and those for whom the invasiveness an RT regimen portends little benefit. Such prognostic factors, particularly those related to biological parameters, would permit development of individualized strategies that lead to improved results by a shift away from radiation-based therapy and toward surgery and/or new target-specific biological agents as more appropriate.

Investigations into the effects of RT and/or chemotherapy on head and neck cancers have tended to focus on genes involved in DNA damage and repair, signal transduction, apoptosis, cell cycle checkpoints and hypoxia. However, the well established heterogeneous response to RT and/or chemotherapy is thought to be due to a very complex interaction of biological parameters that lead to tumor development, growth and invasiveness. Consequently, it is unlikely that individual genes or functionally related groups of genes will have strong predictive power, thereby stressing the need for a more global approach. The development of DNA microarrays has made such a global approach feasible, and has led to some very promising results for other malignancies, such as breast cancers, non-Hodgkin lymphomas, lung adenocarcinomas and esophageal carcinomas. Here, we report for the first time the predictive power of gene expression profiling for the differentiation between controlled and not controlled head and neck squamous cell carcinomas (hnSCC).

### REFERENCES

1. Chiesa, F. et al. Surfing prognostic factors in head and neck cancer at the millennium. Oral Oncol 35, 590-6. (1999).

2 Bataini, J. P., Bernier, J., Jaulerry, C., Brunin, F. & Pontvert, D. Impact of cervical disease and its definitive radiotherapeutic management on survival: experience in 2013 patients with squamous cell carcinomas of the oropharynx and pharyngolarynx. Laryngoscope 100, 716-23. (1990).

3. Allal, A. S. et al. Combined concomitant boost radiotherapy and chemotherapy in stage III-IV head and neck carcinomas: a comparison of toxicity and treatment results with those observed after radiotherapy alone. Ann Oncol 8, 681-4. (1997).

4. Harari, P. M. & Huang, S. M. Head and neck cancer as a clinical model for molecular targeting of therapy: combining EGFR blockade with radiation. Int J Radiat Oncol Biol Phys 49, 427-33. (2001).

5. Begg, A. C. et al. The predictive value of cell kinetic measurements in a European trial of accelerated fractionation in advanced head and neck tumors: an interim report. Int J Radiat Oncol Biol Phys 19, 1449-53. (1990).

6. Koong, A. C. et al. Candidate genes for the hypoxic tumor phenotype. Cancer Res 60, 883-7. (2000).

7. Huang, S. M. & Harari, P. M. Modulation of radiation response after epidermal growth factor receptor blockade in squamous cell carcinomas: inhibition of damage repair, cell cycle kinetics, and tumor angiogenesis. Clin Cancer Res 6, 2166-74. (2000).

8. Allal, A. S. et al. Standardized uptake value of 2-[(18)F] fluoro-2-deoxy-D-glucose in predicting outcome in head and neck carcinomas treated by radiotherapy with or without chemotherapy. J Clin Oncol 20, 1398-404. (2002).

9. Gallo, O. et al. Cumulative prognostic value of p53 mutations and bcl-2 protein expression in head-and-neck cancer treated by radiotherapy. Int J Cancer 84, 573-9. (1999).

10. Wennerberg, J. Predicting response to therapy of squamous cell carcinoma of the head and neck (review). Anticancer Res 16, 2389-96. (1996).

11. Hedenfalk, I. et al. Gene-expression profiles in hereditary breast cancer. N Engl J Med 344, 539-48. (2001).

12. Perou, C. M. et al. Molecular portraits of human breast tumours. Nature 406, 747-52. (2000).

13. van't Veer, L. J. et al. Gene expression profiling predicts clinical outcome of breast cancer. Nature 415, 530-6. (2002).

14. Shipp, M. A. et al. Diffuse large B-cell lymphoma outcome prediction by gene-expression profiling and supervised machine learning. Nat Med 8, 68-74. (2002).

15. Kihara, C. et al. Prediction of sensitivity of esophageal tumors to adjuvant chemotherapy by cDNA microarray analysis of gene-expression profiles. Cancer Res 61, 6474-9. (2001).

16. Eisen, M. B., Spellman, P. T., Brown, P. O. & Botstein, D. Cluster analysis and display of genome-wide expression patterns. Proc Natl Acad Sci USA 95, 14863-8. (1998).

17. Belbin, T. J. et al. Molecular classification of head and neck squamous cell carcinoma using cDNA microarrays. Cancer Res 62, 1184-90. (2002).

18. Hanna, E. et al. A novel alternative approach for prediction of radiation response of squamous cell carcinoma of head and neck. Cancer Res 61, 2376-80. (2001).

### SUMMARY OF THE INVENTION

Radiation therapy (RT) is used extensively in cancer treatment but individual tumors display wide ranges of radio-sensitivity and the outcome for the patient is uncertain. This can be the situation as well in patients with oropharyngeal squamous cell carcinomas. An object of the present invention is to provide a means for assessing responsiveness or susceptibility of oropharyngeal squamous cell carcinoma to radiotherapy in human patients. With this object in mind, the gene expression profiles of tumor biopsies samples from patients with oropharyngeal squamous cell carcinomas were determined. Twelve biopsies samples were from patients whose tumors were cured subsequent to RT were considered susceptible (controlled) profiles. Ten biopsies samples were taken from patients whose carcinomas that were not cured subsequent to RT were considered unsusceptible (not-controlled) profiles. All tumor biopsy samples were analyzed by a standard gene expression procedure utilizing micro arrays containing 4132 human gene sequences. A selection of 738 genes permitted the hierarchical clustering of the biopsies according to their oncological outcome: controlled versus not-controlled.

To confirm the prognostic value of the present hierarchical clustering of the expression profiles of tumor biopsies on the oncological outcome RT treatment of a specific SCC tumor, a blind test was conducted on four additional SCC tumor biopsies. In the blind test, the present system correctly predicted the oncological outcome for each case. Selected gene expression profiling thus provides a tool for predicting the clinical outcome of RT for patients with OROPHARYNGEAL squamous cell carcinoma.

The present system for assessing the susceptibility or responsiveness of a human or-pharyngeal squamous cell carcinoma to radiotherapy is based on the idea that the over-expression and/or under-expression of certain genes in a sample of tumor tissue is predictive of that tumor's to RT. The present invention comprises a selection of genes for use in a gene expression procedure. The selection of genes is challenged with a test sample prepared from a biopsy sample of the carcinoma. The test sample preparation made from a tissue sample taken from the tumor of the patient being assessed. The resulting under- or over-expression certain of genes within the selection of genes in response to the test sample permits an assessment of the susceptibility of the carcinoma to radiotherapy. Gene expression procedures practicable in the present invention are known to one of ordinary skill in the art.

In the present system, the selection of genes were disposed on a gene expression microarray. The microarray may include genes in addition to the present selection of genes. However, it was found that in a selection of genes, the over-expression or under-expression of the gene FLJ11342, H08808, Topoisomerase 1, Dihydrolipoamide dehydrogenase, and EIF4A2 were strongly predictive of oncological outcome after RT.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A to 1C depict a hierarchical clustering of a gene expression analysis of 22 oROPHARYNGEAL SCC tissue samples.

FIG. 1A is an illustration of an experiment tree of raw data from 738 significant genes from the analysis.

FIG. 1B illustrates a gene tree of centered data from 156 selected genes from FIG. 1A.

FIG. 1C further illustrates the experiment tree of centered array and enlarged portion of the gene tree of FIG. 1B (marked in red). Each gene is labeled by its gene name or accession number from GenBank. Green indicates under-expression, red over-expression and black no change compared to the mean, and C=controlled and NC=not controlled.

FIGS. 2A and 2B illustrate a two-dimensional clustering of 4 test biopsies and the 22 oropharyngeal SCC samples. FIG. 2A illustrates the experiment and gene tree of centered data from 173 selected genes. FIG. 2B illustrates an enlarged portion of the gene tree (marked in red). Each gene is labeled by its gene name or accession number from GenBank. Green indicates under-expression, red over-expression and black no change compared to the mean, and C=controlled, NC not controlled, and T=test biopsy sample.

FIG. 3 is an enlarged portion of FIG. 2A illustrating each gene's name or accession number from GenBank.

### DETAILED DESCRIPTION OF THE INVENTION

The present invention is a system for testing susceptibility or responsiveness of a human oropharyngeal squamous cell carcinoma to radiotherapy. Referring now to the drawings, the details of preferred embodiments of the present invention are graphically and schematically illustrated. As illustrated in the figures, the present invention comprises a selection of genes for use in a gene expression procedure. In a preferred embodiment, the selection of genes were disposed on a micro array membrane component of a commercially available gene profiling system. GF211 Human ‘Named Genes’ GeneFilters® Micro arrays—Release 1, Invitrogen Corp. http://www.resgen.com.

The micro array was challenged in the expression procedure with samples of the SCC tumors for which the oncological outcome of RT was later determined. The expression profiles of the over 4000 human gene cDNAs on the micro array membrane were then analyzed to determine gene factor predictive of oncological outcome after patient treatment with RT. Results of the analysis yielded a selection of genes the expression of which was indicative of susceptibility of the tumors tested to RT. Discovery and elucidation of such a selection of genes permits application of the gene expression procedure on a test sample from a patient to provide an assessment of the susceptibility of the test sample's source carcinoma to RT.

To discover genes useful for predicting patient outcome after a radical treatment based on RT, an experimental tumor sample was taken from each of twenty-two patients having the same tumor sublocalization: SCC of the oropharynx. The tumor biopsies samples were taken before any RT treatment. The patients were then subjected to a uniform RT dose and had a minimum of one year follow-up. Post-treatment, patients were separated into two groups according to their oncological outcome: a controlled group (C: 12 patients without any “event”) and a group not-controlled (NC: 10 patients presenting with any “event”). Six patients from the latter group had locoregional failures (2 concomitant with distant metastases) and 4 had distant metastases (Table 1). Tumor persistence or recurrence at the initial primary tumor location and/or at the regional lymph nodes as well as distant metastases were considered as events.

TABLE 1
Patient and Tumor Characteristics

| | | | | | | | | | | |
|---|---|---|---|---|---|---|---|---|---|---|
| | | UICC | Histol. | | Type of | RT dose | Site of | Follow-up | Patients | Cause |
| Age | Sex | stage | Diff. | Treatment | RT | (Gy) | failure | (months) | status | of death |

C

| | | | | | | | | | |
|---|---|---|---|---|---|---|---|---|---|
| 52 | M | 4 | Well | RT | hyperf | 74.40 | 35 | Alive | |
| 53 | F | 4 | Moderate | RT + CHT | Acc | 69.90 | 38 | Alive | |
| 62 | M | 4 | Moderate | RT | Acc | 69.90 | 40 | Alive | |
| 57 | F | 4 | Moderate | RT + CHT | Acc | 69.90 | 12 | Dead | Other |
| 48 | M | 4 | Poor | RT + CHT + ND | Acc | 69.90 | 21 | Alive | |
| 69 | M | 3 | Poor | RT | Acc | 69.90 | 22 | Alive | |
| 44 | M | 3 | Moderate | RT | hyperf | 74.40 | 28 | Alive | |
| 53 | M | 4 | Moderate | RT + CHT | hyperf | 74.40 | 23 | Alive | |
| 73 | M | 3 | Well | RT | Acc | 69.90 | 37 | Alive | |
| 54 | F | 4 | Moderate | RT + CHT | Acc | 69.90 | 25 | Alive | |
| 70 | M | 3 | Well | RT | hyperf | 74.40 | 31 | Alive | |
| 64 | M | 4 | Moderate | RT + CHT | Acc | 70.20 | 39 | Alive | |

NC

| | | | | | | | | | | |
|---|---|---|---|---|---|---|---|---|---|---|
| 51 | M | 4 | Well | RT | hyperf | 74.40 | L | 28 | Alive | |
| 53 | M | 4 | Moderate | RT | hyperf | 74.40 | L + R | 15 | Dead | Cancer |
| 75 | M | 3 | Well | RT | Monof + BT | 68.90 | L + R | 7 | Dead | Cancer |
| 69 | M | 4 | Moderate | RT | Acc | 69.90 | D | 16 | Dead | Cancer |
| 58 | M | 4 | Well | RT | Acc | 69.90 | D | 18 | Dead | Cancer |
| 59 | F | 4 | Moderate | RT + ND | Acc | 69.90 | D | 22 | Dead | Cancer |
| 64 | M | 4 | Poor | RT | Acc | 69.90 | D | 3 | Dead | Cancer |
| 63 | M | 4 | Moderate | RT + CHT | Acc | 69.90 | L + D | 13 | Alive | |
| 58 | M | 3 | Moderate | RT | hyperf | 74.40 | R | 19 | Alive | |
| 52 | F | 4 | Well | RT + CHT | Acc | 69.90 | R + D | 7 | Dead | Cancer |

T

| | | | | | | | | |
|---|---|---|---|---|---|---|---|---|
| 57 | F | 3 | NS | RT + CHT | Acc | 69.90 | 13 | Alive |
| 70 | M | 4 | Moderate | RT | Hyperf | 74.40 | 19 | Alive |
| 79 | M | 4 | Poor | RT | Acc | 69.90 | 12 | Alive |
| 73 | M | 4 | Poor | RT + CHT | Acc | 69.90 | 14 | Alive |

### EXAMPLE I

### Patients

Patient Selection

The study was approved by a local ethical committee. Patients with oropharyngeal squamous cell carcinomas (SCC) satisfying the inclusion criteria were enrolled in the study after signed informed consent was obtained. Pretreatment biopsies were taken prospectively at the time of diagnostic panendoscopy and frozen immediately in liquid nitrogen. Patients included were those treated primarily by RT, either alone or combined with chemotherapy. Patients having had major surgery to the primary tumor area were excluded, whereas patients having only neck dissection were included. The pre-treatment work-up included in all patients a physical examination, a panendoscopy, a magnetic resonance image (MRI) or CT-scan of the head and neck area and in some instances a FDG-PET scanning, and thorax radiography. All tumors were staged according to the 1997-UICC TNM staging system.

Patient Treatment & Follow-Up

A total twenty-two patients were treated with RT. Fourteen of the patients were treated with a modified concomitant-boost accelerated RT schedule that has been reported previously. Another seven patients received hyperfractionated RT to a total dose of 74.4 Gy in 62 fractions over 44 days. One additional patient received monofractionated RT and brachytherapy boost (50.4 Gy+18.5 Gy). The median tumor dose for all patients was 69.9 Gy (range, 68.9-74.4 Gy). All patients were treated with 6 MV photon beams. RT was completed to the planned dose in all patients. Eight of the twenty-two patients received concomitant chemotherapy.

The experimental protocol called for performance of a standard head and neck examination at intervals of one, two, three and six months for the first post-treatment year; every six months for the second, third and fourth post-treatment years, and annually thereafter. Persistent or recurrent tumor was documented by at least two different examinations (MRI or CT-scanner, and endoscopy) and distant metastasis by appropriate tests. While loco-regional failures were generally histologically confirmed, distant metastases were not. The median follow-up for the controlled group was 29 months (range: 12-39 months) and for the uncontrolled group was 15 months (range: 3-28 months).

### EXAMPLE II

### Materials & Methods

Tumor biopsy samples were ground in liquid nitrogen and total RNA isolated with the RNeasy kit (Qiagen, Valencia, Calif.). DNase digestion of the purified RNA was performed for 30 min. at 37° C. with 2 U RQ1 DNase (Promega, Madison, Wis.) per 40 μg of RNA. After addition of 1/10 vol. 20 mM EGTA, pH 8.0, the proteins were removed by two phenol-chloroform extractions. All RNA had an A_{260/280} reading between 1.7 and 2.1 and a 28S/18S ratio of at least 1.4.

Ten μg of total RNA from each sample were converted into ³³P-labeled first-strand cDNA by reverse transcription. The RNA was denatured with 2 μg oligo(dT) for 10 min at 70° C. Then, 300 U Superscript II (Invitrogen, Paisley, Scotland), 6 μl first strand buffer, 3 mM DTT, 3 μl Strip-EZ dNTP (Ambion, Austin, Tex.), and 85 μCi [³³P]dATP (Amersham, Dübendorf, Switzerland) were added to a final volume of 30 μl and incubated for 90 min at 37° C. The probes were freed of unincorporated [³³P]dATP on Bio-Spin 30 columns (Bio-Rad, Hercules, Calif.) and counted in a scintillation counter.

Genefilter membranes GF211 (ResGen, Huntsville, Ala.) were prehybridized for 2 hr at 42° C. with 7.5 μg of denatured Cot-1 DNA and 7.5 μg polyA in 15 ml of MicroHyb hybridization solution. Hybridization was performed for 18 hours at 42° C. with the denatured probe. The membranes were washed twice with 30 ml 2×SSC, 1% SDS for 20 min at 50° C., and once with 0.5×SSC, 0.1% SDS for 15 min at 55° C. Then the membranes were exposed to a phosphor imaging screen (BioRad). Images were acquired with the Personal Molecular Imager FX (Bio-Rad) with a resolution of 50 μ, and analyzed by ImaGene 4.2 software (BioDiscovery, Los Angeles, Calif.). The membranes were stripped with the Strip-EZ RT kit.

### EXAMPLE III

### Cluster and Statistical Analysis

The companion softwares Cluster and Treeview from Eisen et al. (Cluster analysis and display of genome-wide expression patterns. Proc Natl Acad Sci USA 95, 14863-8 (1998)) were used for the bioinformatics calculations. The raw data were log transformed, and complete linkage hierarchical clustering with centered correlation and a cutoff of 0.6 for both arrays and genes was applied. To study differential gene expression, uncentered correlation together with a filter of gene vectors greater than 0.5 was used after genes and arrays were mean centered. For the statistical analysis paired t-test was used.

### EXAMPLE IV

### Hierarchical Clustering of Tumor Sample Expression

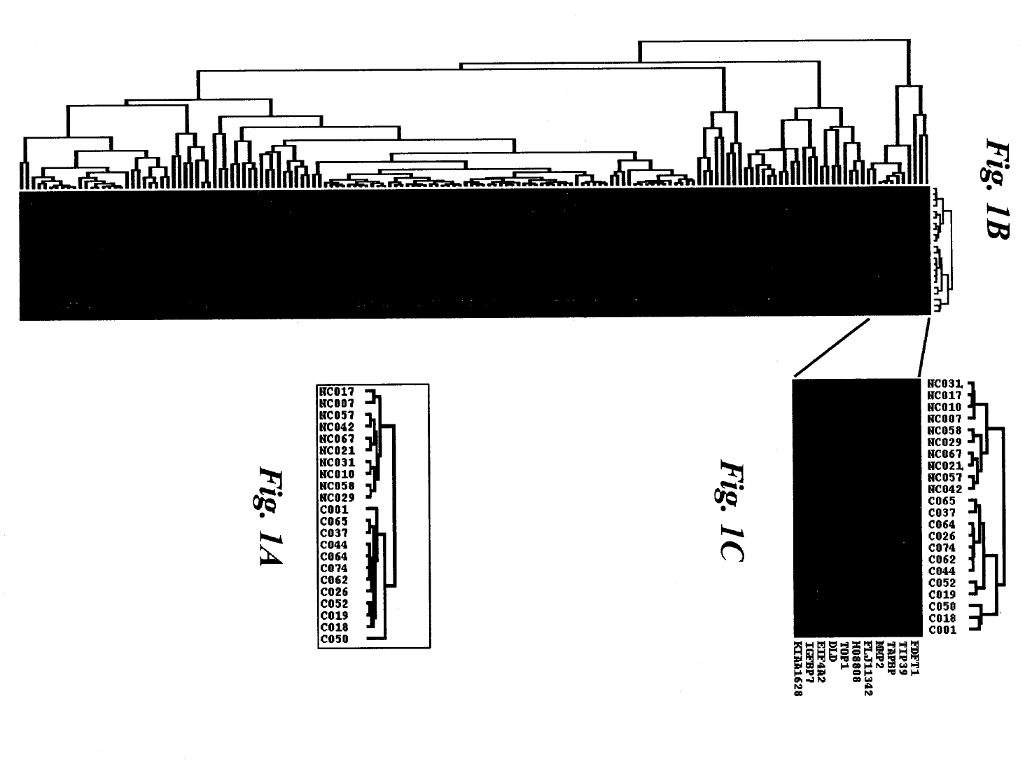

To define biological parameters for the two groups of treatment results, the gene expression profile of each biopsy sample was obtained using a membrane microarrays containing 4132 cDNAs of human genes. Analysis of all 4132 genes from the array did not permit the biopsies to be resolved into the two groups. However, as shown in FIG. 1A-1C, a selection of 738 genes expressing signal over background ratios of greater than 2 enabled the clustering of the samples into the two distinct groups, C and NC, according to their oncological outcome. Clustering was performed in two ways. In the first way, the raw data were used for unsupervised hierarchical clustering (FIG. 1A). In the second way, genes and arrays were mean centered and gene vectors with a standard deviation greater than 0.5 were selected. These stringent criteria filtered 156 genes whose expression profiles also permitted clustering of the biopsies into the two groups (see FIGS. 1B and 1C). The branch lengths reflect the degree of similarity between the tumors or the genes as noted in Eisen et al. above.

The profiles that best discriminate between C and NC are those from the first cluster of 11 genes (gene tree in red, FIG. 1B). Within this cluster, 5 genes (FLJ11342, H08808, TOP1, DLD and EIF4A2) showed a particularly distinct pattern with all controlled tumor biopsies being under-expressed and all not-controlled tumor samples being over-expressed (see FIG. 1C). FLJ1342 and HO8808 are a hypothetical protein and an EST, respectively, with unknown functions. Topoisomerase 1 produces transient single-strand nicks to permit DNA rotation during replication. Dihydrolipoamide dehydrogenase is a protein whose yeast homolog is involved in G1/S cell cycle progression. The translation initiation factor EIF4A2 is a RNA helicase.

The cluster analysis did not permit the differentiation between tumors developing metastases and those recurring locoregionally, perhaps because of the low number of respective biopsies (4 each). Our findings can however be used as an indicator of tumor aggressiveness in general and identify patients with a high probability of recurrence.

### EXAMPLE V

### Prognostic Evaluation

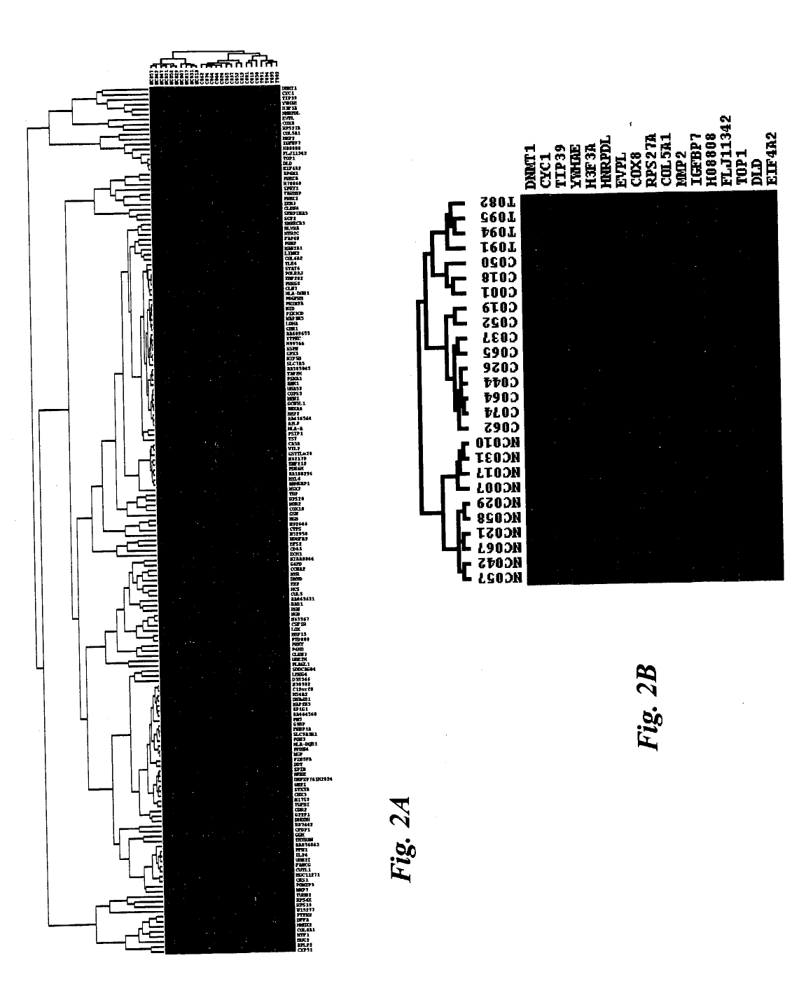

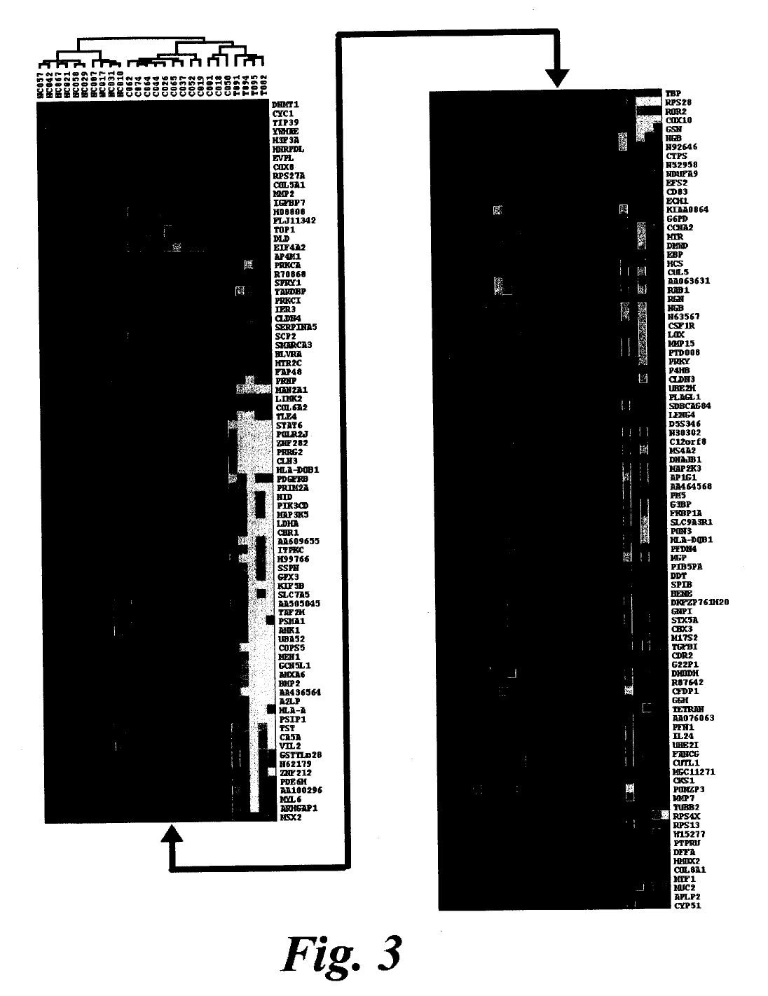

To test the predictive potential of these data, a blind test was performed on four additional biopsies samples from the same oropharyngeal localization by unsupervised hierarchical clustering after mean centering the data. A subset of genes with a standard deviation greater than 0.5 was used in the analysis. This led to the selection of 173 genes, among which the 156 from the previous analysis were present. As shown in FIGS. 2A and 2B, all four test biopsies samples (T) could be accurately clustered with the controlled group. Moreover, the expression pattern of the 5 genes described in FIG. 1C again correlated with the eventual oncological outcome (see FIG. 2B). FIG. 3 is a partial enlargement of FIG. 2A illustrating the gene selection of 173 genes.

The result of this test evaluation indicates that the present system for testing response of a human oropharyngeal squamous cell carcinoma to radiotherapy can in fact predict the clinical outcome for patients subsequently treated with RT (with or without chemotherapy).

While the above description contains many specifics, these should not be construed as limitations on the scope of the invention, but rather as exemplifications of one or another preferred embodiment thereof. Many other variations are possible, which would be obvious to one skilled in the art. Accordingly, the scope of the invention should be determined by the scope of the appended claims and their equivalents, and not just by the embodiments.

What is claimed is:

1. A system for testing susceptibility of a human oropharyngeal squamous cell carcinoma to radiotherapy comprising:
a selection of genes for use in a gene expression procedure with a sample of the carcinoma, which selection of genes permits application of the expression procedure on the sample to provide an assessment of the susceptibility of the carcinoma to radiotherapy.

2. The system of claim 1, further comprising a test preparation made from the sample, the test preparation for use in the expression procedure with the selection of genes.

3. The system of claim 1, wherein the sample causes sufficient expression of genes of the selection of genes, which expression is predictive of susceptibility of the carcinoma to radiotherapy.

4. The system of claim 1, wherein the sample fails to cause sufficient expression of genes of the selection of genes, which failure of sufficient expression is predictive of susceptibility of the carcinoma to radiotherapy.

5. The system of claim 1, wherein the gene expression procedure is known to one of ordinary skill in the art.

6. The system of claim 1, wherein the selection of genes are disposed on a gene expression microarray.

7. The system of claim 1, wherein the selection of genes are a subset of genes disposed on a gene expression microarray.

8. The system of claim 1, wherein the selection of genes includes at least one gene fragment selected from the gene group consisting of FLJ11342, HO8808, Topoisomerase 1, Dihydrolipoamide dehydrogenase, and EIF4A2.

9. A gene expression procedure for testing susceptibility of a human oropharyngeal squamous cell carcinoma comprising the steps of:
providing a gene expression procedure including the genes FLJ11342, H08808, TOP1, DLD and EIF4A2;
taking a sample of the carcinoma to be tested;
preparing a test sample from the carcinoma sample for use in the gene expression procedure;
running the gene expression procedure; and
analyzing the result of the gene expression procedure to predict the whether the tumor is susceptible to radiotherapy.

10. The gene expression procedure of claim 9, wherein the preparing step comprises:
grinding the test sample, digestion of the test sample, radio-labeling of the test sample, hybridization of the test sample, imaging of the test sample and stripping of the test sample.
